(19) United States
(12) Patent Application Publication
(10) Pub. No.: US 2006/0172346 A1
(43) Pub. Date: Aug. 3, 2006

(54) Methods of designing and producing novel compounds having improved binding affinity for CD154 or other trimeric proteins

(76) Inventors: Zhongli Zheng, Lexington, MA (US); Frederick R. Taylor, Milton, MA (US); Christopher D. Benjamin, Beverly, MA (US); Yen-Ming Hsu, Lexington, MA (US)

Correspondence Address: FISH & NEAVE IP GROUP;ROPES & GRAY LLP, 1251 AVENUE OF THE AMERICAS FL C3, NEW YORK, NY, 10020-1105, US

(21) Appl. No.: 11/317,536

(22) Filed: Dec. 23, 2005

Related U.S. Application Data
(63) Continuation of application No. 10/377,688, filed on Feb. 28, 2003, now abandoned.
Application No. 10/377,688 is a continuation of application No. PCT/US01/27360, filed on Aug. 31, 2001.
(60) Provisional application No. 60/230,242, filed on Sep. 1, 2000.
(60) Provisional application No. 60/230,280, filed on Sep. 1, 2000.

(51) Int. Cl. G01N 33/567; C07K 16/28

(52) U.S. Cl. 435/7.21; 530/388.22

(57) ABSTRACT

The present invention relates to methods for designing and producing novel CD40:CD154 binding interrupter compounds and methods using these compounds to treat conditions associated with inappropriate CD154 activation in a subject. This invention also relates to novel methods for screening candidate compounds for the properties of specifically binding CD154 and interrupting CD40:CD154 interaction. This invention further relates to methods of improving the binding affinity, or the ability to block CD40/CD154 interaction, of a synthetic molecule for a trimeric protein, such as CD154. These methods comprise converting a first synthetic molecule that cannot promote lattice or aggregate formation of its target trimeric protein to a second synthetic molecule that can promote lattice or aggregate formation of its target trimeric protein. This invention also relates to a two-dimensional lattice or aggregate comprising a plurality of trimeric CD154 on a cell surface or in solution, wherein the lattice or aggregate is formed by bivalent anti-CD154 antibodies associated with trimeric CD154 molecules.

This application is a continuation of United states application Ser. No. 10/377,688, filed Feb. 28, 2003, which is a continuation of PCT application No. PCT/US01/27360, filed Aug. 31, 2001, which claims benefit of U.S. provisional application No. 60/230,242, filed Sep. 1, 2000 and of U.S. provisional application No. 60/230,280, filed Sep. 1, 2000. The disclosure of United states application Ser. No. 10/377,688 is hereby incorporated by reference herein.

### TECHNICAL FIELD OF THE INVENTION

The present invention relates to methods for designing and producing novel CD40:CD154 binding interrupter compounds and methods using these compounds to treat conditions associated with inappropriate CD154 activation in a subject. This invention also relates to novel methods for screening candidate compounds for the properties of specifically binding CD154 and interrupting CD40:CD154 interaction. This invention further relates to methods of improving the binding affinity, or the ability to block CD40/CD154 interaction, of a synthetic molecule for a trimeric protein, such as CD154. These methods comprise converting a first synthetic molecule that cannot promote lattice or aggregate formation of its target trimeric protein to a second synthetic molecule that can promote lattice or aggregate formation of its target trimeric protein. This invention also relates to a two-dimensional lattice or aggregate comprising a plurality of trimeric CD154 on a cell surface or in solution, wherein the lattice or aggregate is formed by bivalent anti-CD154 antibodies associated with trimeric CD154 molecules.

### BACKGROUND OF THE INVENTION

Data establishing that T cell activation requires both T cell receptor (“TCR”) mediated signals and simultaneously delivered costimulatory signals have accumulated over the past twenty years. For example, antibody production by B lymphocytes in response to protein antigens requires a specific, costimulatory interaction with T lymphocytes. This B cell/T cell interaction is mediated through several receptor-ligand binding events in addition to engagement of the TCR. See, e.g., Noelle et al. Immunology Today 13: 431-433 (1992). See also Hollenbaugh et al. EMBO J. 11: 4313-4321 (1992). These additional binding events include the binding of CD40 on B cells to CD154 (CD40L, and also known as gp39, T-BAM, 5c8 antigen, CD40CR and TRAP) on T cells.

Human CD40 is a 50 kilodalton cell surface protein expressed on mature B cells, as well as macrophages, dendritic cells, fibroblasts and activated endothelial cells. CD40 belongs to a class of receptors involved in cell signaling and programmed cell death, including Fas/CD95 and the tumor necrosis factor alpha (TNF-α) receptor. Human CD154, a 32 kilodalton type II membrane glycoprotein having homology to TNF-α, is a member of the TNF family of receptors and is transiently expressed primarily on activated T cells.

CD40:CD154 binding has been shown to be required for T cell-dependent antibody responses. In particular, CD40:CD154 binding provides anti-apoptotic and/or lymphokine stimulatory signals. CD154, like other TNF family proteins, forms a trimer (i.e., a trimer of CD154 polypeptides). See, e.g., Karpusas et al. Structure 15, 1021-1039 (1995), U.S. patent application Ser. No. 09/180,209 and PCT patent application WO97/00895, the disclosures of which are hereby incorporated by reference.

The importance of CD40:CD154 binding in promoting T cell dependent biological responses is underscored by the development of X-linked hyper-IgM syndrome (X-HIGM) in humans lacking functional CD154. These individuals have normal or high IgM levels, but fail to produce IgG, IgA or IgE antibodies. Affected individuals suffer from recurrent, sometimes severe, bacterial infection (most commonly Streptococcus pneumoniae, Pneumocystis carinii and Hemophilus influenzae) and certain unusual parasitic infections, as well as an increased incidence of lymphomas and abdominal cancers. These clinical manifestations of disease can be managed through intravenous immunoglobulin replacement therapy.

The effects of X-HIGM are simulated in animals rendered nullizygous for the gene encoding CD154 (knockout animals). Studies with nullizygotes have confirmed that, while B cells can produce IgM in the absence of CD40:CD154 binding, they are unable to undergo isotype switching, or to survive normally and undergo affinity maturation. In the absence of a functional CD40:CD154 interaction, spleen and lymph node germinal centers do not develop properly, and the development of memory B cells is impaired. These defects contribute to a severe reduction in or absence of a secondary (mature) antibody response.

Individuals with X-HIGM and CD154 nullizygotes also have defects in cellular immunity. These defects are manifested by an increased incidence of Pneumocystis carinii, Histoplasma capsulatum, Cryptococcus neoformans infection, as well as chronic Giardia lambli infection. Murine nullizygotes are deficient in their ability to fight Leishmania infection. Many of these cell-mediated defects are reversible by administration of IL-12 or IFN-gamma. These data substantiate the view that CD40:CD154 binding promotes the development of Type I T-helper cell responses. This view is further supported by the observation that macrophage activation is defective in CD154-deficient settings, and that administration of anti-CD154 antibodies to mice diminished their ability to clear Pneumocystis infection. Blockade of CD40:CD154 binding appears to reduce the ability of macrophages to produce nitric oxide, which mediates many of the macrophage's pro-inflammatory activities. It should be noted, however, that mammals (including humans) who lack functional CD154 do not develop significant incidences of viral infection.

A number of preclinical studies, including those described in co-pending, commonly assigned PCT patent applications WO98/30241, WO98/30240, WO98/52606, WO98/58669 and WO99/45958, describe the promise of agents capable of interrupting CD40:CD154 binding as immunomodulating agents. In murine systems, antibodies to CD154 block primary and secondary immune responses to exogenous antigens, both in vitro and in vivo. Antibodies to CD154 cause a reduction in germinal centers in mice and monkeys, consistent with data on CD154 immunodeficiency. Administration of three doses of anti-CD154 antibody to lupus-prone mice, age three months, substantially reduced titers against double-stranded DNA and nucleosomes, delayed the development of severe nephritis, and reduced mortality. Moreover, administration of anti-CD154 antibodies to mice age five to seven months with severe nephritis was shown to stabilize or even reverse renal disease. Anti-CD154 antibodies given concomitantly with small resting allogeneic lymphocytes permitted unlimited survival of mouse pancreatic islet allografts. In other animal models, interference with CD40:CD154 binding has been demonstrated to reduce symptoms of autoimmune disease (e.g., multiple sclerosis, rheumatoid arthritis, inflammatory bowel disease), graft rejection (e.g., cardiac allograft, graft-versus-host disease), and mercuric chloride induced glomerulonephritis, which is mediated by both humoral and cellular mechanisms.

Such studies with anti-CD154 antibodies demonstrate the role of CD154 as a critical target for modulating immune responses.

Novel synthetic compounds that bind to CD154 or to another target trimeric protein, such as another member of the TNF family, may be screened by, inter alia, the following methods: random screening of libraries of chemical compounds that bind to the target protein; or by obtaining structural information of a complex of the target protein, preferably with an agent that is known to bind specifically to that protein, by X-ray crystallography and subsequent use of computational means to rationally design and ultimately make synthetic compounds. These compounds, however, may or may not bind with high affinity to their target protein(s). The most desired of these compounds as candidates for human therapeutic agents would be those which bind with high affinity to their target protein(s).

### SUMMARY OF THE INVENTION

The present invention provides methods for designing and making novel CD40:CD154 binding interruptor compounds. This invention also provides an assay to detect whether an agent that specifically binds CD154 promotes lattice or aggregate formation of CD154 trimers on a cell surface or in solution. This assay may be used as a method to screen for novel CD40:CD154 binding interrupter compounds that specifically bind CD154 and interrupt its interaction with CD40.

This invention further relates to methods for improving the binding affinity or ability to block CD40:CD154 interaction of a synthetic molecule for a trimeric protein, wherein the synthetic molecule specifically binds to the trimeric protein but with low affinity (with a Kd in the micromolar range or higher), is a poor blocker of CD40/CD154 interaction, or both. Such methods result in converting a synthetic molecule which does not significantly promote lattice or aggregate formation of trimers of CD154 on a cell surface or in solution to one that does. Lattice or aggregate formation enhances the potency of a molecule by the large-scale cooperativity of the binding event. The lattice or aggregate can also have the effect of making potential binding sites of the protein for its receptor inaccessible by sequestering the protein in a large complex. This sequestration results even if the molecule does not bind at a site on the protein that directly blocks its interaction with its receptor.

This invention further relates to compositions comprising the novel CD40:CD154 binding interrupter compounds. The invention further relates to methods of using these novel compounds to treat a subject with, inter alia, one or more conditions associated with inappropriate CD154 activation. This invention further relates to methods using these novel compounds to treat a subject having one or more conditions associated with expression of a trimeric protein other than CD154.

This invention also relates to a two-dimensional lattice or aggregate comprising a plurality of trimeric CD154 molecules on a cell surface or in solution, wherein the lattice or aggregate is formed by anti-CD154 antibodies cross-linking the trimeric CD154 molecules.

The foregoing and other objects, features and advantages of the present invention, as well as the invention itself, will be more fully understood from the following description of preferred embodiments.

### BRIEF DESCRIPTION OF THE DRAWINGS

FIG. 1. Confocal microscope image of the mAb 5c8-induced clustering of CD154 molecules on the surface of a cell and the lack of clustering by TRAP-1 mAb. Stained in light grey: anti-mouse Cy5, which stained murine mAb 5c8 (a murine anti-human CD154 monoclonal antibody) or TRAP-1. Murine mAb 5c8 is referred to as mu5c8. Syto13 DNA/RNA counterstain (image for TRAP-1 not shown) was also used. MAb 5c8 staining was polarized into a cap-like structure on the cell surface (shown in Cy5 stain in light grey), indicating that CD154 had been rearranged into a lattice or aggregate. MAb TRAP-1 staining (shown in Cy5 stain in light grey) was evenly dispersed over the entire cell surface, indicating no large-scale CD154 rearrangement as observed for mAb 5c8 staining, although small-scale rearrangement of CD154 trimers might exist. DNA/RNA counterstain was dispersed over the entire cell (shown only for mAb 5c8). This DNA/RNA counterstain cannot be properly visualized in this black and white version of this figure.

FIG. 2. Gel filtration chromatographic analysis of mAb 5c8-CD154 complexes.

FIG. 3. Light scatter analysis of mAb 5c8-CD154 complexes.

### DETAILED DESCRIPTION OF THE INVENTION

The TNF Family of Proteins

CD154 belongs to the tumor necrosis factor (TNF) protein family. The family members include, inter alia, TNF-α, TNF-β (also known as lymphotoxin a or “LTa”), lymphotoxin β (“LTβ”), Fas ligand, CD30L, CD27L and 4-1BBL. Members of this family are critically involved in the regulation of inflammation, immune response and tissue homeostasis. See, e.g., Smith et al. Cell 76: 959-962 (1994) and Vassalli Ann. Rev. Immunolol. 10: 411-452 (1992), the disclosures of which are hereby incorporated by reference. Most if not all of the proteins of this family form trimers. The pharmaceutical importance of members of the TNF family is underscored by FDA approval and the commercial sale of two drugs, REMICADE™ and EMBREL®, which target the TNF pathway.

An agent, such as a synthetic compound, that binds a trimeric protein, such as a TNF family protein, may bind with low affinity (with a Kd in the micromolar range or higher). In one embodiment of this invention, the binding affinity of this agent for that trimeric protein may be improved by designing and making an improved version of the agent by converting it from one that does not cross-link trimers of that protein into one that does. Lattice or aggregate formation enhances the potency of a molecule by the large-scale cooperativity of the binding event. The lattice or aggregate can also have the effect of making potential binding sites of the protein for its receptor inaccessible by sequestering the protein in a large complex. This sequestration results even if the molecule does not bind at a site on the protein that directly blocks its interaction with its receptor. In a preferred embodiment of this invention, the trimeric protein is a TNF family protein. In a more preferred embodiment, the TNF family protein is CD154.

Synthetic Compounds

Synthetic compounds (molecules) that bind to CD154 or another trimeric protein, such as another TNF family member, may be obtained by, inter alia, the following screening methods: randomly screening libraries of chemical compounds that bind to a particular protein; or by obtaining structural information of a complex of the protein and an agent that is known to bind specifically to that protein by X-ray crystallography and subsequent use of computational means to rationally design and ultimately make synthetic compounds. Some of the synthetic compounds may bind their target protein with low affinity and may not be able to cross-link cell-surface bound trimers of their target protein and thereby promote a plurality of said trimers on a cell surface or in solution to form a lattice or aggregate.

Methods for Designing and Producing Compounds Having Improved Binding Affinity for a Trimeric Protein

In a preferred embodiment, this invention provides a method for improving the binding affinity of a first synthetic molecule for a trimeric protein by generating a second synthetic molecule therewith, comprising the steps of:
(a) providing a first synthetic molecule that binds a trimeric protein with low affinity and is not capable of cross-linking a plurality of said trimeric protein and thereby promoting a plurality of said trimeric protein on a cell surface or in solution to form a lattice or aggregate;
(b) providing a means to generate a second synthetic molecule comprising said first synthetic molecule, wherein said second synthetic molecule is capable of cross-linking a plurality of said trimeric protein and promoting a plurality of said trimeric protein on a cell surface or in solution to form a lattice or aggregate; and
(c) generating said second synthetic molecule based on said means.

In a preferred embodiment, the trimeric protein is a protein other than CD154. In a more preferred embodiment, the trimeric protein is a TNF family member other than CD154.

The cell can be any cell that expresses a trimeric protein or can be made to express a trimeric protein on its cell surface (for example, by transfecting into a cell a vector comprising the gene of a trimeric protein under the control of a suitable promoter/enhancer). In a preferred embodiment, the cell is a mammalian cell. In the case where the trimeric protein is CD154, the cell could be a T lymphocyte or an immortalized T cell. The immortalized T cell can be a D1.1 cell derived from ATCC Accession No. CRL 10915 (deposited on Nov. 14, 1991).

In another more preferred embodiment, the trimeric protein is CD154, and, more preferably, human CD154.

In a more preferred embodiment, the first synthetic molecule specifically binds trimeric CD154. In another more preferred embodiment, the first synthetic molecule specifically binds trimeric 5c8 antigen, which is specifically bound by monoclonal antibody 5c8 (ATCC Accession No. HB 10916, deposited on Nov. 14, 1991). 5c8 antigen is human CD154. A human CD154 DNA sequence and a human CD154 amino acid sequence were disclosed in Hollenbaugh et al., EMBO J., 11: 4313-4321 (1992).

In another preferred embodiment, the first synthetic molecule inhibits a process selected from the group consisting of T cell dependent B cell activation, B cell dependent T cell activation, humoral immune response and cellular immune response.

In a preferred embodiment, the second synthetic molecule is a tethered bivalent molecule.

In another preferred embodiment, the second synthetic molecule comprises a pair of binding moieties linked via an organic linker arm. In a more preferred embodiment, each member of the pair of binding moieties of the second synthetic molecule binds specifically to trimeric CD154.

In another preferred embodiment, the second synthetic molecule comprises a pair of binding moieties, each of which binds specifically to trimeric 5c8 antigen, which is specifically bound by monoclonal antibody 5c8 (produced by the hybridoma having ATCC Accession No. HB 10916).

The second synthetic molecule of this invention retains the binding specificity of the first synthetic molecule from which the second synthetic molecule is converted.

A person skilled in the art would know how to produce a second synthetic molecule that cross-links trimers of a given protein from a first synthetic molecule without that property. That person would know how to use a number of conventional methods to design and make the second synthetic molecules. For example, such methods include organic synthesis and computational fitting.

Methods for Designing and Producing Novel CD40:CD154 Binding Interruptor Compounds and Assays to Detect Promotion of Lattice or Aggregate Formation of CD154 Trimers by an Agent that Specifically Binds CD154

CD154 forms a trimer. The membrane-bound form of CD154 or CD154 in solution is almost exclusively trimeric, which is the biologically functional form.

In a preferred embodiment, a method is provided for screening a candidate compound for the property of specifically binding to CD154 and interrupting CD40:CD154 interaction, said method comprising the steps of:
(a) providing a cell having trimeric CD154 molecules on the cell surface;
(b) incubating said cell with said candidate compound, under conditions sufficient for said compound to promote cross-linking of said CD154 molecules if said compound has the property of promoting such cross-linking; so as to produce a lattice or aggregate of CD154 molecules on the cell surface;
(c) incubating said cell with a detectable agent that specifically binds to cell surface CD154 molecules; and
(d) detecting that a lattice or aggregate of cross-linked CD154 molecules has formed on the cell surface.

In another preferred embodiment, a method is provided for screening a candidate compound for the property of specifically binding to CD154 and interrupting CD40:CD154 interaction, said method comprising the steps of:
(a) providing a solution comprising trimeric CD154 molecules;
(b) incubating said solution with said candidate compound, under conditions sufficient for said compound to promote cross-linking of said trimeric CD154 molecules in solution if said compound has the property of promoting such cross-linking; so as to produce a lattice or aggregate of CD154 molecules in solution; and
(c) detecting that a lattice or aggregate of cross-linked CD154 molecules has formed in solution.

An assay is provided by this invention for screening a candidate compound for the property of specifically binding CD154 and interrupting CD40:CD154 interaction. The candidate compound is either an anti-CD154 antibody or a synthetic compound defined herein.

One assay detects the promotion of lattice or aggregate formation of cell-surface CD154 trimers by an agent (that agent may be an antibody or a synthetic compound defined herein) that specifically binds CD154. Any assay that can detect lattice or aggregate formation of cell-surface CD154 trimers by an agent that specifically binds CD154 may be used. An example of such an assay is a cell-binding assay.

As shown in Example 1, as detected in a cell binding assay, monoclonal antibody (“mAb”) 5c8, which specifically binds human CD154, promotes capping (lattice or aggregate formation) of a plurality of trimeric human CD154 on a cell surface by cross-linking CD154 trimers.

In a cell binding assay, a detectable agent that specifically binds to cell surface CD154 molecules is added to CD154 molecules on a cell surface and formation of a lattice or aggregate of cross-linked CD154 molecules on the cell surface is detected by microscopy. An example of a detectable agent is an anti-CD154 antibody or a synthetic compound directly conjugated with a fluorochrome, which fluoresces under a certain wave-length. Another example of a detectable agent requires a first reagent, which is an anti-CD154 antibody or a synthetic compound that specifically binds CD154, and a secondary reagent, such as a secondary antibody (e.g. a goat anti-mouse IgG Fab fragment as shown in Example 1), protein A, protein G or strepavidin (provided that the anti-CD154 antibody or synthetic compound is biotinylated). The secondary reagent is conjugated with a fluorochrome and binds to the anti-CD154 antibody or the synthetic compound that specifically binds CD154.

The cell can be any cell that expresses CD154 or can be made to express CD154 on its cell surface (for example, by transfecting into a cell a vector comprising a CD154 gene under the control of a suitable promoter/enhancer). In a preferred embodiment, the cell is a mammalian cell. The cell can be a T lymphocyte or an immortalized T cell. The immortalized T cell can be a D1.1 cell derived from ATCC Accession No. CRL 10915 (deposited on Nov. 14, 1991).

In an alternative embodiment, another assay is provided by this invention for screening a candidate compound for the property of specifically binding CD154 and interrupting CD40:CD154 interaction. This assay detects the promotion of lattice or aggregate formation of CD154 trimers in solution by a candidate compound (which may be an antibody or a synthetic compound defined herein) that specifically binds CD154. The ability of the candidate compound to induce aggregation of CD154 in solution using a soluble form of CD154 can be detected. The type of aggregate formed in solution will depend on the binding site of the antibody or synthetic compound on CD154 and the length and flexibility of linker connecting the bivalent compounds. In some cases, large scale aggregates will be formed, analogous to immune precipitates, and these are visible to the eye or readily detected by a reduction in light transmission over a broad span of wavelengths. In other cases, smaller-scale complexes may form, particularly when the linker is of sufficient length and flexibility to allow the formation of caged structures. Such structures can be visualized using several techniques, including gel filtration, light scatter, electron microscopy and fluorescence energy transfer.

In a gel filtration assay, as shown in Example 4, soluble human CD154 and mAb 5c8 or humanized mAb 5c8 are mixed and allowed to bind. The mixture is then chromatographed on a suitable gel filtration column, such as the Superose 6 (Pharmacia). The elution of the proteins is monitored at 280 nm.

In a light scattering assay, as shown in Example 5, soluble human CD154 and mAb 5c8 or humanized 5c8 mAb are mixed together and allowed to bind. The mixture is then subjected to light scatter analysis on a detector, such as the Precision Detector PDLS 2000 instrument.

If an antibody or a synthetic compound is shown to promote lattice or aggregate formation by any of the above-identified assays, it is preferable to confirm that the antibody or synthetic compound does indeed interrupt CD40:CD154 interaction, as the property of an agent capable of promoting lattice or aggregate formation of CD154 trimers on a cell surface is correlated with that agent's ability to interrupt CD40:CD154 interaction. This confirmatory step can be performed by any assay that detects CD40:CD154 interaction.

A person skilled in the art is aware of conventional assays to assess whether the compounds designed according to this invention bind specifically to CD154 and whether such compounds interrupt CD40:CD154 interaction. Some of these assays detect whether, or the extent to which, B cells are activated by activated T cells via the interaction between CD154 and CD40. For example, monitoring of CD23 levels on B cells, or secretion of immunoglobulins by B cells is indicative of activation of B cells by activated T cells via the interaction between CD40 and CD154. Accordingly, these assays are, for example, in vitro assays for T cell activation of B cells; in vitro assays for immunoglobulin production by B cells and in vivo assays for inhibition of a humoral immune response. See, e.g., U.S. Pat. No. 5,474,771. Accordingly, examples of such assays include: an in vitro assay for T cell activation of B cells, an in vitro assay for immunoglobulin production by B cells and an in vivo assay for inhibition of a humoral immune response.

Lattice or Aggregate

In a preferred embodiment, this invention provides a multi-molecular, two-dimensional lattice or aggregate comprising:
(a) a plurality of trimeric CD154 molecules disposed on the extracellular surface of a cell membrane (preferably a mammalian cell membrane) or in solution; and
(b) anti-CD154 monoclonal antibody molecules, or antigen-binding fragments thereof, each cross-linking said plurality of trimeric CD154 molecules such that a lattice or aggregate is formed on the extra-cellular surface of said cell membrane or in solution.

The cell membrane can be of any cell that expresses CD154 or can be made to express CD154 on its cell surface (for example, by transfecting into a cell a vector comprising a CD154 gene under the control of a suitable promoter/enhancer). In a preferred embodiment, the cell is a mammalian cell. The cell can be a T lymphocyte or an immortalized T cell. The immortalized T cell can be a D1.1 cell derived from ATCC Accession No. CRL 10915, described in, for example, U.S. Pat. No. 5,474,771 (deposited on Nov. 14, 1991).

Lattice or aggregate formation enhances the potency of a molecule by the large-scale cooperativity of the binding event. The lattice or aggregate can also have the effect of making potential binding sites of the protein (such as CD154) for its receptor (such as CD40) inaccessible by sequestering the protein in a large complex. This sequestration results even if the molecule does not bind at a site on the protein that directly blocks its interaction with its receptor.

In a more preferred embodiment, the anti-CD154 monoclonal antibody, or antigen-binding fragment thereof, of this multi-molecular lattice or aggregate interrupts the binding of CD154 to CD40.

In another more preferred embodiment, the CD154 monoclonal antibody specifically binds to the 5c8 antigen, which is specifically bound by monoclonal antibody 5c8 (ATCC Accession No. HB 10916).

Example 1 illustrates the capping (i.e., promotion of lattice or aggregate formation) of human CD154 on the surface of Jurkat D1.1 cells (having by the hybridoma having ATCC Accession No. CRL 10915) by mAb 5c8 but not by mAb TRAP-1.

Antibodies that Specifically Bind CD154

Antibodies that specifically bind CD154 (anti-CD154 antibodies) include polyclonal antibodies and monoclonal antibodies (mAbs), as well as antibody derivatives such as chimeric molecules, humanized antibodies, antibodies with altered (e.g., reduced) effector functions, bispecific molecules, and conjugates of antibodies. Preferably, the antibody is mAb 5c8 (produced by the hybridoma having ATCC Accession Number HB 10916), as described in U.S. Pat. No. 5,474,771, the disclosure of which is hereby incorporated by reference. In a highly preferred embodiment, the antibody is a humanized 5c8 mAb. Other known antibodies against human CD154 include antibodies ImxM90, ImxM91 and ImxM92 (described in U.S. Pat. No. 5,961,974). Numerous additional anti-human CD154 antibodies have been produced and characterized (see, e.g., PCT patent application WO96/23071 of Bristol-Myers Squibb, the specification of which is hereby incorporated by reference). For lattice or aggregate formation involving murine systems, antibodies which specifically bind to murine CD154 should be used—an example of such an antibody is MR1 (see Noelle et al. (1992), Proc. Natl. Acad. Sci. USA 89: 6550). The selection of an appropriate monoclonal antibody (mAb) will depend on the species from which the CD154 is derived and the species specificity of the anti-CD154 monoclonal antibody. For example, mAb 5c8, produced by the hybridoma having ATCC Accession No. HB 10916 and raised against human CD154, specifically binds to human and some non-human primate CD154 molecules but not to murine CD154 and it should therefore be selected for human and some non-human primate use and not murine use.

In a preferred embodiment, the antibody, or an antigen-binding fragment thereof, binds specifically to human CD154. Examples include mAb 5c8, humanized mAb 5c8, and Fab′, (Fab)₂ and Fab fragments of mAb 5c8 or humanized mAb 5c8.

The invention also includes anti-CD154 antibodies of other types, such as single chain antibodies (see, e.g., PCT patent application WO96/23071).

Various forms of antibodies may also be produced using standard recombinant DNA techniques (Winter and Milstein, Nature 349: 293-99, 1991). For example, “chimeric” antibodies may be constructed, in which the antigen binding domain from an animal antibody is linked to a human constant domain (an antibody derived initially from a nonhuman mammal in which recombinant DNA technology has been used to replace all or part of the hinge and constant regions of the heavy chain and/or the constant region of the light chain, with corresponding regions from a human immunoglobulin light chain or heavy chain) (see, e.g., Cabilly et al., U.S. Pat. No. 4,816,567; Morrison et al., Proc. Natl. Acad. Sci. 81: 6851-55, 1984). Chimeric antibodies reduce the immunogenic responses elicited by animal antibodies when used in human clinical treatments.

In addition, recombinant “humanized” antibodies may be synthesized. Humanized antibodies are antibodies initially derived from a nonhuman mammal in which recombinant DNA technology has been used to substitute some or all of the amino acids not required for antigen binding with amino acids from corresponding regions of a human immunoglobulin light or heavy chain. That is, they are chimeras comprising mostly human immunoglobulin sequences into which the regions responsible for specific antigen-binding have been inserted (see, e.g., PCT patent application WO94/04679). Animals are immunized with the desired antigen, the corresponding antibodies are isolated and the portion of the variable region sequences responsible for specific antigen binding are removed. The animal-derived antigen binding regions are then cloned into the appropriate position of the human antibody genes in which the antigen binding regions have been deleted. Humanized antibodies minimize the use of heterologous (inter-species) sequences in antibodies for use in human therapies, and are less likely to elicit unwanted immune responses. Primatized antibodies can be produced similarly using primate (e.g., rhesus, baboon and chimpanzee) antibody genes.

Another embodiment of the invention includes the use of human antibodies, which can be produced in nonhuman animals, such as transgenic animals harboring one or more human immunoglobulin transgenes. Such animals may be used as a source for splenocytes for producing hybridomas, as described in U.S. Pat. No. 5,569,825.

Univalent antibodies may also be used in the methods of this invention. Univalent antibodies comprise a heavy chain/light chain dimer bound to the Fc (or stem) region of a second heavy chain.

In addition, standard recombinant DNA techniques can be used to alter the binding affinities of recombinant antibodies with their antigens by altering amino acid residues in the vicinity of the antigen binding sites. The antigen binding affinity of a humanized antibody may be increased by mutagenesis based on molecular modeling (Queen et al., Proc. Natl. Acad. Sci. 86:10029-33, 1989; PCT patent application WO94/04679). It may be desirable to increase or to decrease the affinity of the antibodies for CD154. This may be done utilizing phage display technology (see, e.g., Winter et al., Ann. Rev. Immunol. 12:433-455, 1994; and Schier et al., J. Mol. Biol. 255:28-43, 1996, which are hereby incorporated by reference).

Conditions Associated with Inappropriate CD154 Induced Activation in a Sublect

The synthetic compounds designed according to this invention can be used to prevent or treat subjects having conditions associated with inappropriate CD154 induced activation. Treating a condition associated with inappropriate CD154 induced activation in a subject includes, inter alia, attenuating severity of the condition, suppressing effects of the condition, inhibiting the condition and reversing the condition.

Examples of conditions associated with inappropriate CD154 mediated activation in a subject, include, inter alia: unwanted immune response, an unwanted inflammatory response, an autoimmune disease, an allergy, an inhibitor response to a therapeutic agent, rejection of a donor organ and a B cell cancer.

Examples of conditions associated with inappropriate CD154 mediated activation in a subject, include, inter alia: systemic lupus erythematosis, lupus nephritis, lupus neuritis, asthma, chronic obstructive pulmonary disease, bronchitis, emphysema, multiple sclerosis, uveitis, Alzheimer's disease, traumatic spinal cord injury, stroke, atherosclerosis, coronary restenosis, ischemic congestive heart failure, cirrhosis, hepatitis C, diabetic nephropathy, glomerulonephritis, osteoarthritis, rheumatoid arthritis, psoriasis, atopic dermatitis, systemic sclerosis, radiation-induced fibrosis, Crohn's disease, ulcerative colitis, multiple myeloma and cachexia.

Subjects

The novel CD40:CD154 binding interrupter compounds designed according to this invention, which can cross-link trimeric CD154 molecules on a cell surface or in solution, can be administered for treatment or prophylaxis to any mammalian subject suffering or about to suffer a condition associated with inappropriate CD154 activation. Preferably, the subject is a primate, more preferably a higher primate, most preferably a human. In other embodiments, the subject may be a mammal of commercial importance, or a companion animal or other animal of value, such as a member of an endangered species. Thus, a subject may be, inter alia, sheep, horses, cattle, goats, pigs, dogs, cats, rabbits, guinea pigs, hamsters, gerbils, rats and mice.

Route of Administration

The CD40:CD154 binding interrupter compounds designed according to this invention may be administered in any manner which is medically acceptable. Depending on the specific circumstances, local or systemic administration may be desirable. Local administration may be, for example, by subconjunctival administration. Preferably, the interrupter is administered via an oral, an enteral, a parenteral route such as by an intravenous, intraarterial, subcutaneous, intramuscular, intraorbital, intraventricular, intraperitoneal, subcapsular, intracranial, intraspinal, topical or intranasal injection, infusion or inhalation. The interruptor also may be administered by implantation of an infusion pump, or a biocompatible or bioerodable sustained release implant, into the subject.

Dosages and Frequency of Treatment

Generally, the methods described herein involve administration of a CD40:CD154 binding interrupter compound designed according to this invention at desired intervals (e.g., daily, twice weekly, weekly, biweekly, monthly or at other intervals as deemed appropriate) over at least a two- or three-week period. The administration schedule is adjusted as needed to treat the condition associated with inappropriate CD154 activation in the subject. The present treatment regime can be repeated in the event of a subsequent episode of illness.

The amount and frequency of dosing for any particular compound to be administered to a patient for a given immunological disease associated with inappropriate CD154 induced activation in a subject is within the skills and clinical judgement of ordinary practitioners of the medical and pharmaceutical arts. The general dosage and administration regime is established by preclinical and clinical trials, which involve extensive but routine studies to determine the optimal administration parameters of the compound. Even after such recommendations are made, the practitioner will often vary these dosages for different subjects based on a variety of considerations, such as the individual's age, medical status, weight, sex, and concurrent treatment with other pharmaceuticals. Determining the optimal dosage and administration regime for each anti-CD154 compound used is a routine matter for those of skill in the medical and pharmaceutical arts.

Generally, the frequency of dosing may be determined by an attending physician or similarly skilled practitioner, and might include periods of greater dosing frequency, such as at daily or weekly intervals, alternating with periods of less frequent dosing, such as at monthly or longer intervals.

To exemplify dosing considerations for a CD40:CD154 binding interrupter, the following examples of administration strategies are given for an anti-CD154 mAb. Such dosing amounts serve as guidelines for the treating physician administering CD40:CD154 binding interrupters according to this invention and could easily be adjusted by the treating physician administering such other types of anti-CD154 compounds. In general, single dosages of between about 0.05 and about 50 mg/kg patient body weight may be used, with dosages most frequently in the 1-20 mg/kg range. For acute treatment, such as before or at the time of transplantation, or in response to any evidence that graft rejection is beginning, an effective dose would be equivalent to that of a representative antibody (such as mAb 5c8), which ranges from about 1 mg/kg body weight to about 20 mg/kg body weight, administered daily for a period of about 1 to 5 days, preferably by bolus intravenous administration. The same dosage and dosing schedule may be used in the load phase of a load-maintenance regimen, with the maintenance phase involving intravenous or intramuscular administration of antibodies in a range of about 0.1 mg/kg body weight to about 20 mg/kg body weight, for a treatment period of anywhere from weekly to 3 month intervals. Chronic treatment may also be carried out by a maintenance regimen patterned on that of antibodies are administered by intravenous or intramuscular route, in a range of about 0.1 mg/kg body weight to about 20 mg/kg body weight, with interdose intervals ranging from about 1 week to about 3 months. In addition, chronic treatment may be effected by an intermittent bolus intravenous regimen, patterned on that of antibodies in which between about 1.0 mg/kg body weight and about 100 mg/kg body weight of antibodies are administered, with the interval between successive treatments being from 1 to 6 months. For all except the intermittent bolus regimen, administration may also be by oral, pulmonary, nasal or subcutaneous routes.

For treatment, a CD40:CD154 binding interrupter compound can be formulated in a pharmaceutical or prophylactic composition which includes, respectively, a pharmaceutically or prophylactically effective amount of the CD40:CD154 binding interrupter dispersed in a pharmaceutically acceptable carrier. In some embodiments, the pharmaceutical or prophylactic composition can also include a pharmaceutically or prophylactically effective amount of another immunosuppressive or immunomodulatory compound, including without limitation: an agent that interrupts T cell costimulatory signaling via CD28 (e.g., CTLA4-Ig), CD80 or CD86; an agent that interrupts calcineurin signaling (e.g., cyclosporin, a macrolide such tacrolimus, formerly known as FK506); a corticosteroid; or an antiproliferative agent (e.g., azathioprine). Other therapeutically effective compounds suitable for use with the CD40:CD154 binding interrupter include rapamycin (also known as sirolimus); mycophenolate mofetil (MMF), mizoribine, deoxyspergualin, brequinar sodium, leflunomide, azaspirane and the like.

Combination therapies according to this invention for treatment of a condition associated with inappropriate CD154 activation in a subject include the use of a CD40:CD154 binding interruptor together with agents targeted at B cells, such as anti-CD19, anti-CD28 or anti-CD20 antibody (unconjugated or radiolabeled), IL-14 antagonists, LJP394 (LaJolla Pharmaceuticals receptor blocker), IR-1116 (Takeda small molecule) and anti-Ig idiotype monoclonal antibodies. Alternatively, the combinations may include T cell/B cell targeted agents, such as CTLA4Ig, IL-2 antagonists, IL-4 antagonists, IL-6 antagonists, receptor antagonists, anti-CD80/CD86 monoclonal antibodies, TNF, LFA1/ICAM antagonists, VLA4/VCAM antagonists, brequinar and IL-2 toxin conjugates (e.g., DAB), prednisone, anti-CD3 mAb (OKT3), mycophenolate mofetil (MMF), cyclophosphamide, and other immunosuppressants such as calcineurin signal blockers, including without limitation, tacrolimus (FK506). Combinations may also include T cell targeted agents, such as CD4 antagonists, CD2 antagonists and anti-IL-12 antibodies.

The immunomodulatory compound that may be co-administered with an CD40:CD154 binding interrupter compound to a subject having a condition associated with inappropriate CD154 activation may be an antibody that specifically binds to a protein selected from the group consisting of CD45, CD2, IL2R, CD4, CD8 and RANK Fc.

Formulation

In general, compounds designed according to the methods of this invention are suspended, dissolved or dispersed in a pharmaceutically acceptable carrier or excipient. The resulting therapeutic composition does not adversely affect the recipient's homeostasis, particularly electrolyte balance. Thus, an exemplary carrier comprises normal physiologic saline (0.15M NaCl, pH 7.0 to 7.4). Other acceptable carriers are well known in the art and are described, for example, in Remington's Pharmaceutical Sciences, Gennaro, ed., Mack Publishing Co., 1990. Acceptable carriers can include biocompatible, inert or bioabsorbable salts, buffering agents, oligo- or polysaccharides, polymers, viscoelastic compound such as hyaluronic acid, viscosity-improving agents, preservatives, and the like.

A CD40:CD154 binding interrupter compound designed using the methods of this invention may be administered in a pharmaceutically effective, prophylactically effective or therapeutically effective amount, which is an amount sufficient to produce a detectable, preferably medically beneficial effect on a subject at risk or afflicted with a condition associated with inappropriate CD154 activation. Medically beneficial effects include preventing, inhibiting, reversing or attenuating deterioration of, or detectably improving, the subject's medical condition.

Synthetic Compounds with Improved Affinity for Trimeric Proteins Other than CD154

Synthetic compounds designed by the methods of this invention with improved affinity for trimeric proteins other than CD154, such as TNF family proteins other than CD154, may be used to treat conditions which respond to such proteins. The subjects, route of administration, dosage and frequency of treatment and formulation for these compounds will depend on the particular protein target to which a particular synthetic compound binds. The skilled artisan would appreciate these nuances and would be able to use these compounds to treat the right subjects by the correct route of administration, dosage and frequency of treatment and formulation without undue experimentation.

### EXAMPLES

The following are examples that illustrate the methods, compounds and compositions of this invention. These examples should not be construed as limiting: the examples are included for the purposes of illustration only.

### Example 1

### A Cell-Binding Assay: Confocal Microscopy Lattice or Aggregate Formation Assay Showing Staining Pattern of Monoclonal Antibodies 5C8 and Trap-1 on D1.1 Cells

MAb 5c8 or TRAP-1, each of which is a murine anti-human CD154 antibody, was bound to D1.1 Jurkat cells at 1 μg/ml for 1 hour at 37° C. in RPMI (media) with 10% FBS to allow for rearrangement of CD154. Cells were fixed in ice-cold 1% paraformaldehyde for 10 minutes to cross-link cell surface proteins and prevent further rearrangement. Cells were incubated on ice with a goat anti-mouse IgG Fab fragment conjugated with the fluorochrome Cy5 for 30 minutes in FACs (PBS with 1% BSA and 0.1% sodium azide) buffer with 2% goat serum to detect CD154-bound mAb 5c8 or mAb TRAP-1. The cells were then incubated with the fluorochrome Syto 13 to counterstain the DNA and RNA. Slides of the cells were viewed using a confocal microscope.

This method can directly detect the anti-CD154 antibody bound to human CD154 on the cell surface. This method can be applied to all murine antibodies and can easily be used for other antibodies, for example, human or humanized antibodies, by for example, using a human specific secondary antibody.

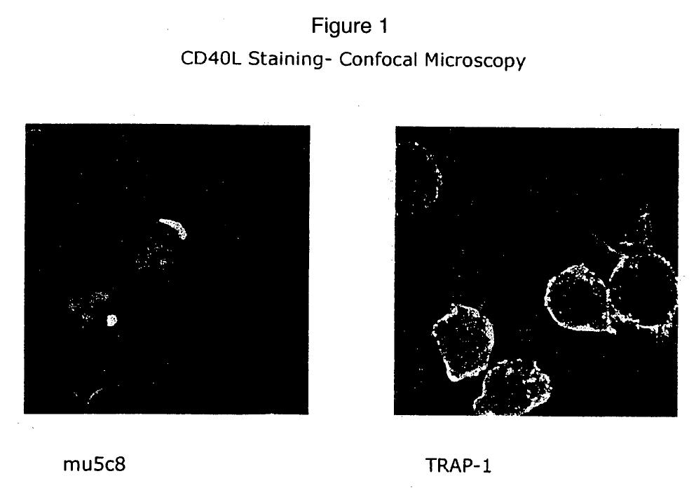

As shown in FIG. 1, mAb 5c8 staining was polarized into a cap-like structure on the cell surface, indicating that CD154 had been rearranged into a lattice or aggregate. MAb TRAP-1 staining was evenly dispersed over the entire cell surface, indicating no large-scale CD154 rearrangement as observed for mAb 5c8 staining, although small-scale rearrangement of CD154 trimers might exist.

### Example 2

### A Gel Filtration Assay

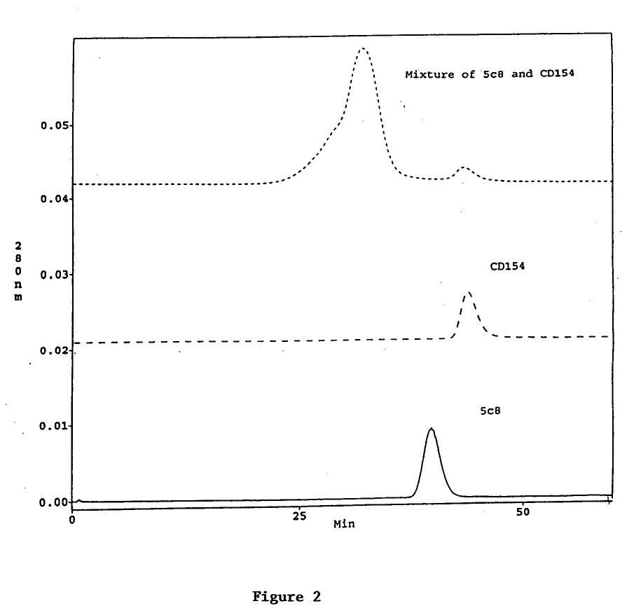

Soluble human CD154 (5.3 mg/ml) and mAb 5c8 (15.2 mg/ml) were mixed together at a 1:1 molar ratio in PBS buffer and allowed to incubate at room temperature over night. The mixture was then chromatographed on a Superose 6 gel filtration column (Pharmacia) in PBS at a flow rate of 0.5 mL/min and the elution of the proteins monitored at 280 nm. A complex was formed between CD154 and mAb 5c8, whose molecular weight, as estimated by its elution time relative to standards, was >800 kDa, indicating that the complex consisted of an aggregate of several mAb 5c8 and CD154 molecules. See FIG. 2.

### Example 3

### A Light Scattering Assay

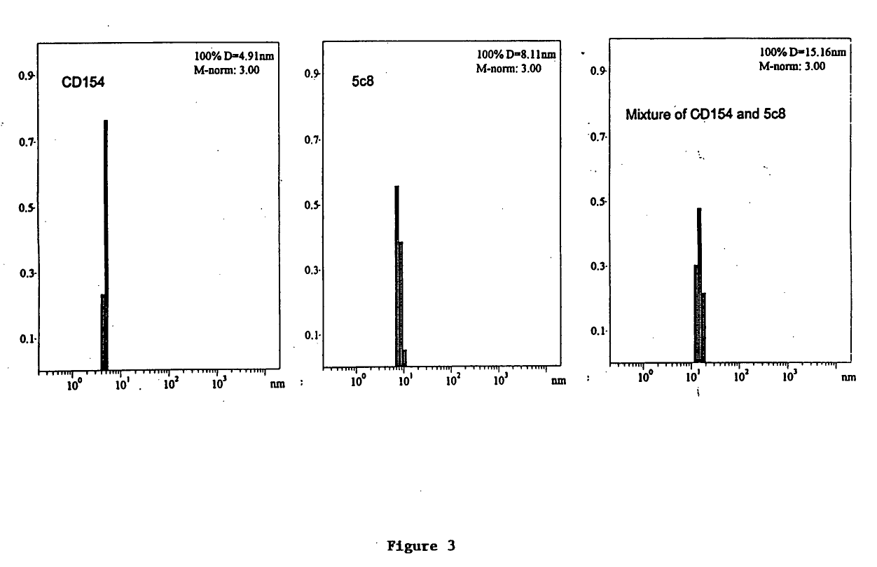

Soluble human CD154 (0.11 mg/ml) and mAb 5c8 (0.5 mg/ml) were mixed together overnight and subjected to light scatter analysis on a Precision Detector PDLS 2000 instrument. A complex was formed between CD154 and mAb 5c8, whose diameter translated to a molecular weight of >800 kDa, indicating that the complex consisted of an aggregate of several mAb 5c8 and CD154 molecules. See FIG. 3.

### EQUIVALENTS

The invention may be embodied in other specific forms without departing from the spirit or essential characteristics thereof. The foregoing embodiments are therefore to be considered in all respects illustrative of, rather than limiting on, the invention disclosed herein. All changes which come within the meaning and range of equivalency of the claims are intended to be embraced therein.

1. A multi-molecular, two-dimensional lattice or aggregate comprising:
(a) a plurality of trimeric CD154 molecules disposed on the extracellular surface of a cell membrane; and
(b) anti-CD154 monoclonal antibody molecules, or antigen-binding fragments thereof, each cross-linking said plurality of trimeric CD154 molecules such that a lattice or aggregate is formed on the extra-cellular surface of said cell membrane.

2. A multi-molecular, two-dimensional lattice or aggregate comprising:
(a) a plurality of trimeric CD154 molecules in solution; and
(b) anti-CD154 monoclonal antibody molecules, or antigen-binding fragments thereof, each cross-linking said plurality of trimeric CD154 molecules such that a lattice or aggregate is formed in solution.

3. The multi-molecular, two-dimensional lattice or aggregate according to claim 1, wherein said cell membrane is a mammalian cell membrane.

4. The multi-molecular lattice or aggregate according to claim 1 or 2, wherein said anti-CD154 monoclonal antibody molecules, or antigen-binding fragments thereof, interrupt the binding of CD154 to CD40.

5. The multi-molecular lattice or aggregate according to claim 1 or 2, wherein said CD154 monoclonal antibody molecules specifically bind to the 5c8 antigen, which is specifically bound by monoclonal antibody 5c8 (produced by the hybridoma having ATCC Accession No. HB 10916).

6. A method for improving the binding affinity of a first synthetic molecule for a trimeric protein by generating a second synthetic molecule therewith, comprising the steps of:
(a) providing a first synthetic molecule that binds a trimeric protein with low affinity and is not capable of cross-linking a plurality of said trimeric protein and thereby promoting a plurality of said trimeric protein on a cell surface to form a lattice or aggregate;
(b) providing a means to generate a second synthetic molecule comprising said first synthetic molecule, wherein said second synthetic molecule is capable of cross-linking a plurality of said trimeric protein and promoting a plurality of said trimeric proteins on a cell surface to form a lattice or aggregate; and
(c) generating said second synthetic molecule based on said means.

7. A method for improving the binding affinity of a first synthetic molecule for a trimeric protein by generating a second synthetic molecule therewith, comprising the steps of:
(a) providing a first synthetic molecule that binds a trimeric protein with low affinity and is not capable of cross-linking a plurality of said trimeric protein and thereby promoting a plurality of said trimeric proteins in solution to form a lattice or aggregate;
(b) providing a means to generate a second synthetic molecule comprising said first synthetic molecule, wherein said second synthetic molecule is capable of cross-linking a plurality of said trimeric protein and promoting a plurality of said trimeric protein in solution to form a lattice or aggregate; and
(c) gene rating said second synthetic molecule based on said means.

8. The method according to claim 6 or 7, wherein said trimeric protein is a protein other than CD154.

9. The method according to claim 8, wherein said trimeric protein is a TNF family member protein.

10. The method according to claim 6 or 7, wherein said trimeric protein is CD154.

11-18. (canceled)

19. A method for screening a candidate compound for the property of interrupting CD40:CD154 interaction, comprising the steps of:
(a) providing a cell having trimeric CD154 molecules on the cell surface;
(b) incubating said cell with said candidate compound, under conditions sufficient for said compound to promote cross-linking of said CD154 molecules if said compound has the property of promoting such cross-linking; so as to produce a lattice or aggregate of CD154 molecules on the cell surface;
(c) incubating said cell with a detectable agent that specifically binds to cell surface CD154 molecules; and
(d) detecting that a lattice or aggregate of cross-linked CD154 molecules has formed on the cell surface.

20. A method for screening a candidate compound for the property of interrupting CD40:CD154 interaction, comprising the steps of:
(a) providing a solution comprising trimeric CD154 molecules;
(b) incubating said solution with said candidate compound, under conditions sufficient for said compound to promote cross-linking of said trimeric CD154 molecules in solution if said compound has the property of promoting such cross-linking; so as to produce a lattice or aggregate of CD154 molecules in solution; and
(c) detecting that a lattice or aggregate of cross-linked CD154 molecules has formed in solution.

21. The met hod according to claim 19, wherein said detectable agent is a monoclonal antibody.

22. The method according to claim 19, wherein said detectable agent is directly linked to a fluorochrome.

23. The method according to claim 19, wherein said detectable agent is indirectly linked to a fluorochrome via a secondary reagent that is linked to a fluorochrome.

24. The method according to claim 19, wherein said cell is a T lymphocyte.

25. The method according to claim 19, wherein said cell is an immortalized T cell.

26. The method according to claim 25, wherein said immortalized T cell is a D1.1 cell derived from ATCC Deposit No. CRL 10915.

27. The method according to claim 19 or claim 20, further comprising the step of:
(e) confirming whether the candidate compound capable of promoting lattice or aggregate formation also is capable of interrupting CD40:CD154 interaction.

28. The method according to claim 27, wherein step (e) is performed using an in vitro assay for T cell activation of B cells.

29. The method according to claim 27, wherein step (e) is performed using an in vitro assay for immunoglobulin production by B cells.

30. The method according to claim 27, wherein step (e) is performed using an in vivo assay for inhibition of a humoral immune response.

31. A method for screening a candidate compound for the property of interrupting CD40:CD154 interaction, comprising the steps of:
(a) providing a cell having trimeric CD154 molecules on the cell surface;
(b) incubating said cell with said candidate compound, under conditions sufficient for said compound to promote cross-linking of said CD154 molecules if said compound is capable of promoting such cross-linking; so as to produce a lattice or aggregate of CD154 molecules on the cell surface; and
(c) detecting that a lattice or aggregate of cross-linked CD154 molecules has formed on the cell surface by a cell-binding assay.

32. A method for screening a candidate compound for the property of interrupting CD40:CD154 interaction, comprising the steps of:
(a) providing a solution comprising trimeric CD154 molecules in solution;
(b) incubating said solution with said candidate compound, under conditions sufficient for said compound to promote cross-linking of said CD154 molecules if said compound is capable of promoting such cross-linking; so as to produce a lattice or aggregate of CD154 molecules in solution; and
(c) detecting that a lattice or aggregate of cross-linked CD154 molecules has formed on the cell surface by an assay selected from the group consisting of gel filtration assay and light scattering assay.

33. The method according to claim 31 or 32, further comprising the step of:
(d) confirming whether the candidate compound capable of promoting lattice or aggregate formation also has the property of interrupting CD40:CD154 interaction.

34-53. (canceled)
